(19) United States
(12) Patent Application Publication
(10) Pub. No.: US 2024/0368663 A1
(43) Pub. Date: Nov. 7, 2024

(54) CELL-FREE BIOPRODUCTION OF B-CRYPTOXANTHIN AND ZEAXANTHIN

(71) Applicant: DEBUT BIOTECHNOLOGY, INC., San Diego, CA (US)

(72) Inventors: DREW WAGNER, San Diego, CA (US); Nicholas Brideau, San Diego, CA (US); Joshua Britton, San Diego, CA (US)

(21) Appl. No.: 18/576,602

(22) PCT Filed: Jul. 19, 2022
(86) PCT No.: PCT/US2022/073874

§ 371 Date: Jan. 4, 2024

Related U.S. Application Data
(60) Provisional application No. 63/223,678, filed on Jul. 20, 2021.

(51) Int. Cl. C12P 23/00; C12N 9/02

(52) U.S. Cl. CPC C12P 23/00; C12N 9/0073; C12Y 114/13129; C12Y 114/18; C12Y 114/19

(57) ABSTRACT

A cell-free system is provided to produce β-cryptoxanthin and zeaxanthin from β-carotene. β-carotene is added to a reaction mixture comprising a β-carotene hydroxylase enzyme (CrtZ). The reaction mixture may be aqueous and may comprise a co-solvent, e.g., an organic co-solvent, such as THF.

### I. RELATED APPLICATIONS

This application claims priority to U.S. Provisional Application No. 63/223,678, filed on Jul. 20, 2021. The content of U.S. Provisional Application No. 63/223,678 is hereby incorporated by reference in its entirety.

### II. SEQUENCE LISTING

This application contains a sequence listing filed in electronic form as an .xml file entitled DEBU-010-01WO-ST26.xml, created on Jul. 14, 2022 and having a size of 12 KB. The content of the sequence listing is incorporated herein its entirety.

### III. FIELD OF THE INVENTION

The present invention features a method of producing carotenoids by enzyme modification of β-carotene and its derivatives. In particular, the present invention features a cell-free production method.

### IV. BACKGROUND OF THE INVENTION

Carotenoids are naturally occurring yellow, orange, and red pigments. There are over 1100 known carotenoids found in plants, algae, bacteria, and fungi. Carotenoids are the source of color for many fruits and vegetables and other animals and plants such as salmon, canaries, shrimp, egg yolks, marigolds, and some autumn leaves. Carotenoids can be further categorized into xanthophylls (which contain oxygen) and carotenes (which are purely hydrocarbons and contain no oxygen).

Alpha-carotene (α-carotene), beta-carotene (β-carotene), beta-cryptoxanthin (β-cryptoxanthin), lutein, zeaxanthin, and lycopene are the most common dietary carotenoids, and many carotenoids are known to have a range of health benefits. For example, β-carotene and β-cryptoxanthin are precursors to Vitamin A (retinol) and they can be converted to the active form of the vitamin for skin and bone health as well as immune function. β-cryptoxanthin has greater bio-availability than β-carotene and has also been shown to have both antioxidant and anti-cancer properties. As another example, zeaxanthin plays a role in protecting the eyes from harmful effects of oxidation and may decrease the risk of macular degeneration. Additionally, zeaxanthin can help cells clear free radicals and protect skin against UV damage.

### V. SUMMARY OF THE INVENTION

Although carotenoids are responsible for the coloration of many consumed foods, the dietary intake of zeaxanthin and β-cryptoxanthin is deficient, and supplementation is needed. As with all carotenoids, the amount of β-cryptoxanthin and zeaxanthin available from cultivation is dependent on seasonal effects such as cultivar, growing conditions, and storage methods. Furthermore, the processing and cooking of fruits and vegetables such as refining, drying, and boiling can destroy carotenoids or severely limit their bioaccessibility. A manufacturing solution outside of cultivation and extraction is needed.

Manufacturing of carotenoids via chemical synthesis or in cells suffers from problems that limit the commercial viability of high-value chemical production. Chemical synthesis requires extensive, elaborate, expensive, toxic, and inefficient multi-step chemical reactions to produce natural products that often are too complex to make in the laboratory. Manufacturing processes involving bio-foundries (use of the whole cell) suffer from product toxicity, carbon flux redirection, diffusion problems through cell walls, and toxic byproduct generation.

To avoid the aforementioned problems, the inventors have developed cell-free manufacturing of carotenoids as a viable alternative to synthetic and bio-foundry manufacturing methods.

In one aspect, the invention provides a method for cell-free production of hydroxylated carotenoid, wherein the method comprises transformation of a substrate through an enzyme to hydroxylated carotenoid. In certain embodiments, the substrate is a carotene. In certain embodiments, the carotene is β-carotene. In certain embodiments, the hydroxylated carotenoid is a β-cryptoxanthin or zeaxanthin. In certain embodiments, the enzyme is selected from a group consisting of: (i) β-carotene hydroxylase or a homolog thereof, (ii) sterol desaturase or a homolog thereof, (ii) fatty acid hydroxylase or a homolog thereof, and (iv) any combination thereof. In certain embodiments, the enzyme is immobilized. In certain embodiments, the enzyme is not immobilized. In certain embodiments, the β-carotene hydroxylase has an amino acid sequence at least 80% identical to the polypeptides set forth in SEQ ID NOS. 1 or 2. In certain embodiments, the enzyme is a sterol desaturase and has an amino acid sequence at least 80% identical to one of the polypeptides set forth in SEQ ID NOS. 3-10. In certain embodiments, the hydroxylated carotenoid is produced at a concentration of at least 500 mg/L. In certain embodiments, the method further comprises the use of a solvent, and the solvent is selected from a group consisting of: (i) tetrahydrofuran (THF), (ii) dimethylsulfoxide (DMSO), (iii) dimethylformamide (DMF), and (iv) any combination thereof. In certain embodiments, the method further comprises the use of one or more non-ionic surfactants and/or detergents. In certain embodiments, the method comprises the use of one or more natural or synthetic reduction-oxidation (redox) couplers. In certain embodiments, the method comprises the use of a continuous reactor system. In certain embodiments, the method comprises the steps of: in a cell-free vessel, providing a β-carotene hydroxylase enzyme (CrtZ); adding β-carotene; removing the hydroxylated carotenoid from the cell-free vessel. In certain embodiments, the hydroxylated carotenoid is a di-hydroxylated carotenoid, and the substrate is a mono-hydroxylated carotenoid.

In another aspect, the invention provides a composition for cell-free production of hydroxylated carotenoid by enzymatic transformation of a substrate to a hydroxylated carotenoid, wherein the composition comprises a substrate and an enzyme. In certain embodiments, the substrate is carotene. In certain embodiments, the carotene is β-carotene. In certain embodiments, the hydroxylated carotenoid is a β-cryptoxanthin or zeaxanthin. In certain embodiments, the enzyme is selected from a group consisting of: (i) β-carotene hydroxylase or a homolog thereof, (ii) sterol desaturase or a homolog thereof, (ii) fatty acid hydroxylase or a homolog thereof, and (iv) any combination thereof. In certain embodiments, the enzyme is immobilized. In certain embodiments, the enzyme is not immobilized. In certain embodiments, the β-carotene hydroxylase has an amino acid sequence at least 80% identical to the polypeptides set forth in SEQ ID NOS. 1 or 2. In certain embodiments, the enzyme is a sterol desaturase and has an amino acid sequence at least 80% identical to one of the polypeptides set forth in SEQ ID NOS. 3-10. In certain embodiments, the hydroxylated carotenoid is produced at a concentration of at least 500 mg/L. In certain embodiments, the composition further comprises a solvent, wherein the solvent is selected from a group consisting of: (i) tetrahydrofuran (THF), (ii) dimethylsulfoxide (DMSO), (iii) dimethylformamide (DMF), and (iv) any combination thereof. In certain embodiments, the composition further comprises one or more non-ionic surfactants and/or detergents. In certain embodiments, the method comprises the use of one or more natural or synthetic reduction-oxidation (redox) couplers. In certain embodiments, the hydroxylated carotenoid is a di-hydroxylated carotenoid, and the substrate is a mono-hydroxylated carotenoid.

In the cell-free systems described herein, the critical components of the cell, namely cofactors and enzymes, are used in a chemical reaction without cellular components that can directly or indirectly inhibit the desired biochemical reaction. The same enzymes found in plants and other organisms may be created in vivo (typically through protein overexpression in hosts such as bacteria), isolated via chromatography, and then added into a bioreactor with a substrate (starting material). The enzymes transform the substrate in the same way that occurs in the original organism without the organism's complexity. Additionally, the biochemical reaction may be enhanced by the addition of co-solvents, detergents, or both, which would not be tolerated by, or simply would not work in a whole cell-based manufacturing method. In this way, natural products can be created without the plant, cell, or chemical synthesis.

The present invention's objective is to provide a cell-free biosynthesis platform that allows for the production of hydroxylated carotenoids (e.g., β-cryptoxanthin and zeaxanthin) starting from carotenes (non-hydroxylated carotenoids), e.g., β-carotene. Additionally, the methods herein may be employed to hydroxylate mono-hydroxylated carotenoids to produce di-hydroxylated carotenoids. Embodiments of the present invention can be freely combined with each other if they are not mutually exclusive.

In some embodiments, the present invention uses β-carotene hydroxylase enzymes to add molecular oxygen to carotenes (e.g., β-carotene) and yield the corresponding mono- or di-hydroxylated carotenoids (e.g., β-cryptoxanthin and zeaxanthin).

The presently claimed process uses a controlled enzymatic step to increase product (mono- or di-hydroxylated carotenoids) titer in short reaction times. In some embodiments, the controlled enzymatic step may increase product titer by a factor of at least about fifteen to about sixty over titers obtained with bio-foundry methods. Additionally, enzyme immobilization may be used to enhance β-carotene conversion to corresponding hydroxylated carotenoids, e.g., β-cryptoxanthin and zeaxanthin.

A unique and inventive technical feature of the present invention is the use of a cell-free system for the production of hydroxylated carotenoids β-cryptoxanthin and zeaxanthin. Without wishing to limit the invention to any theory or mechanism, it is believed that the technical feature of the present invention advantageously provides for higher reaction concentrations of one or more of starting materials (substrates, e.g., β-carotene and β-cryptoxanthin), reagents, and/or enzymes, resulting in higher concentrations of final products (e.g., β-cryptoxanthin and zeaxanthin). Additionally, the present invention eliminates the complications of cell walls, thereby eliminating a significant barrier to product and substrate diffusion. Furthermore, the present invention eliminates competition for carbon flux, which limits the efficiency of cell-based synthesis methods, and thus greatly reduces byproduct formation. Also, because there is no cell, the present invention is not vulnerable to degradation by cell death due to the formation of toxic compounds. In addition, the present methods enhance the ability to use various solvents, such as organic solvents, to permit higher concentrations of solutes (e.g., substrates and intermediates) without worrying about killing the cell.

The present invention is adaptable to multiple products. In this approach, one simply changes the starting carotenoid (e.g., β-carotene or β-cryptoxanthin) in the pathway to synthesize the desired hydroxylated carotenoid product. In some embodiments, β-carotene may be mono-hydroxylated to form β-cryptoxanthin or di-hydroxylated to zeaxanthin. The same enzyme (CrtZ) may be used to either mono- or di-hydroxylate the various carotenoids. The degree of hydroxylation may be adjusted by varying the reaction time, the co-solvent type, concentration, or both, varying the detergent, detergent concentration, or both, varying the substrate (β-carotene or mono-hydroxylated β-carotene) starting concentration, or a combination of two or more thereof. In general, shorter reaction times favor mono-hydroxylation of β-carotene substrate, while longer reaction times favor di-hydroxylation.

Furthermore, the previously known methods teach away from the present invention. The maximum titers achievable for the current cell-based production of β-cryptoxanthin and zeaxanthin are limited. Previously published work involving extensive cellular reprogramming and metabolic engineering produced the carotenoid zeaxanthin on the scale of β-50 mg/L. The present invention affords a production titer of at least 1000 mg/L of zeaxanthin, representing a 20-fold increase from reported values. In certain embodiments of the current invention, the present invention affords a production titer of at least about 1500 mg/L, about 2000 mg/L, about 2500 mg/L, and about 3000 mg/L. These embodiments of the invention represent a 30-fold, 40-fold, 50-fold, or 60-fold increase respectively from the previously reported values.

Additionally, the starting material β-carotene and products β-cryptoxanthin and zeaxanthin are insoluble in water. Addition of a co-solvent such as tetrahydrofuran (THF), dimethylsulfoxide (DMSO) dimethylformamide (DMF) (or mixtures of two or more thereof) greatly improves the solubility and stability of the carotenoids, which the present invention permits. Some methods, according to the present invention, can operate at co-solvent levels>15% (v/v), which is much higher than cell-based carotenoid production, as living cells are sensitive to most co-solvent concentrations above 5%.

An additional advantage of the current invention over prior art is the use of non-ionic surfactants and other detergents to solubilize substrate, products, and the cellular membrane fractions containing the CrtZ enzyme. Addition of non-ionic surfactants such as, but not limited to, Polysorbate 20, Triton X-100, Tween-80, and NP-40 significantly increase the activity of CrtZ in the acellular reaction system. Gram-negative bacteria such as E. coli are naturally resistant to solubilization by detergents at low concentrations and are sensitive to detergents at high concentrations, making product extraction from cells difficult. Some methods of the present invention operate at non-ionic surfactant levels up to 10% (v/v), e.g., about 0.01% (v/v) to about 10% (v/v), about 0.1% (v/v) to about 10% (v/v), about 1% (v/v) to about 10% (v/v) or about 2% (v/v) to about 10% (v/v). See definition herein for “about X % (v/v),” wherein X is a variable from 0 to 100.

An additional advantage of the current invention over prior art is the use of electron transfer reagents, also referred to as redox couplers, to enhance substrate flux to the CrtZ enzyme. Addition of electron transfer reagents significantly increases the activity of CrtZ in the acellular reaction system. The examples of suitable electron transfer reagents include methyl viologen, phenazine methosulfate, 2,6-dichloro-1,4-benzoquinone and diethyldithiocarbamate. Gram-negative bacteria such as E. coli are incompatible with the use of such electron transfer reagents and are sensitive to electron transfer reagents at even moderate concentrations, making their use limited in cell-based reactions. Some methods of the present invention operate at synthetic electron transfer reagents levels up to about 100 μM. In certain preferred embodiments of the invention, the electron transfer reagents are present at a concentration of about 1 μM to about 100 μM.

The inventive technical features of the present invention contributed to a further unexpected result of the selective conversion of β-carotene to either β-cryptoxanthin or zeaxanthin. The β-carotene hydroxylase CrtZ can perform both mono- and di-hydroxylation of β-carotene, and the process in cells rarely stops at the single hydroxylation event. Selecting appropriate cell-free reaction conditions can favor the production of either β-cryptoxanthin or zeaxanthin depending on the variables that are changed, as described in more detail herein. For example, varying solvent type and percentage, detergent type and concentration, reaction duration, and substrate concentration, or a combination of the above, are all viable methods to produce β-cryptoxanthin over zeaxanthin selectively.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### VI. BRIEF DESCRIPTION OF DRAWINGS

FIG. 1 shows the chemical transformation pathway for the production of β-cryptoxanthin and zeaxanthin from β-carotene.

FIGS. 2A-2C show HPLC traces for β-carotene, β-cryptoxanthin, zeaxanthin standards (FIG. 2A), and enzyme reactions favoring zeaxanthin (FIG. 2B) and β-cryptoxanthin (FIG. 2C). The retention times of 7.2 minutes (zeaxanthin) and 12.2 minutes (β-cryptoxanthin) and 16.9 minutes (β-carotene) at 450 nm are noted. The HPLC conditions (temperature, stationary and mobile phases) are described herein.

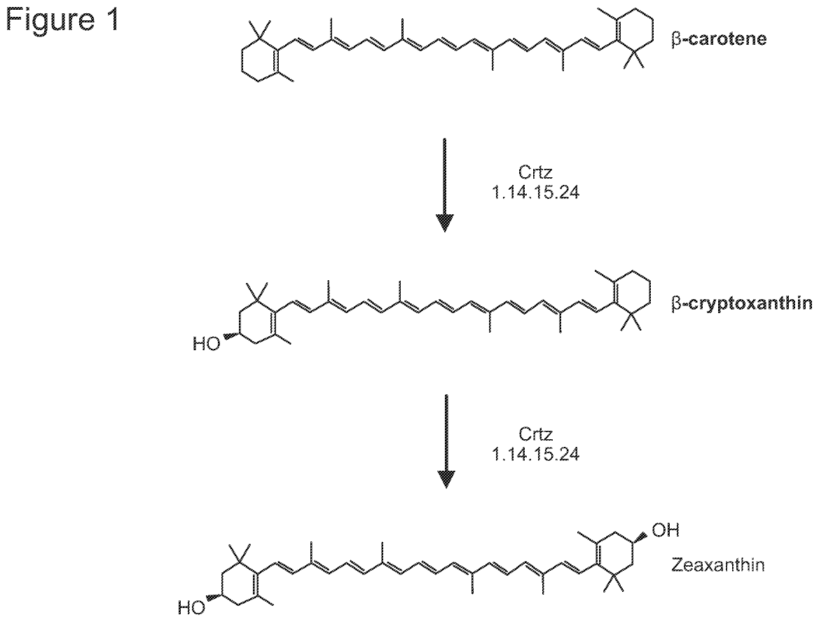

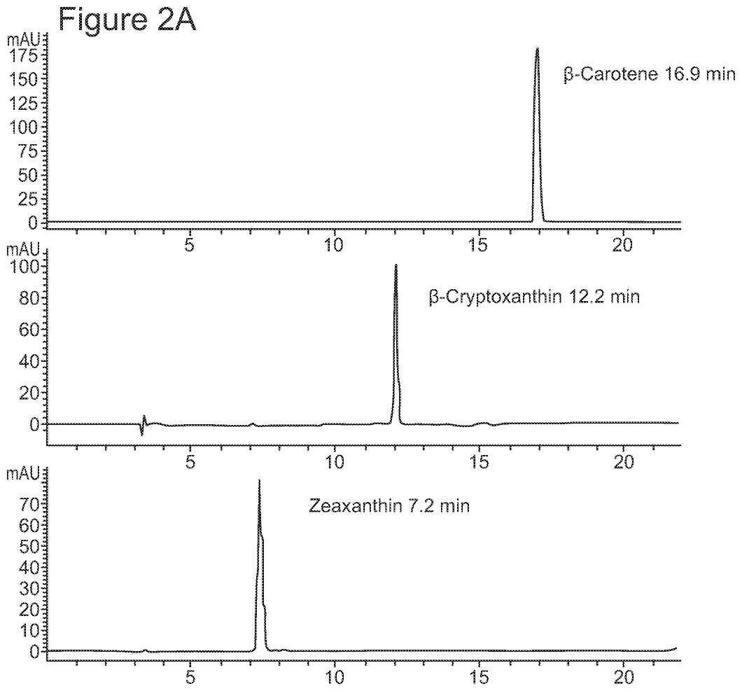

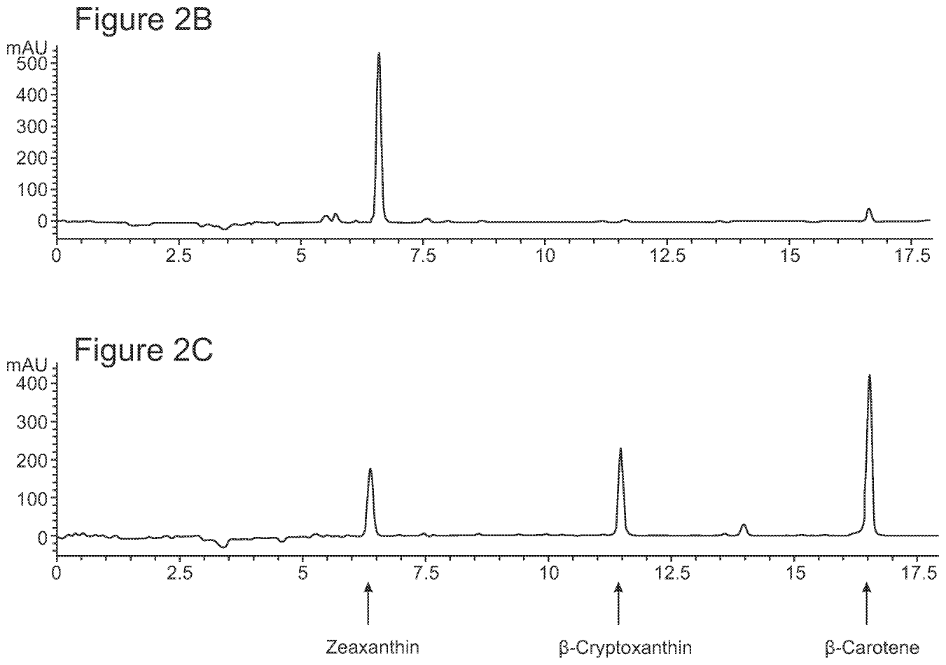

### VII. DETAILED DESCRIPTION OF THE INVENTION

Before the present compounds, compositions, and/or methods are disclosed and described, it is to be understood that, unless specified, this invention is not limited to specific synthetic methods or to specific compositions, as such may, of course, vary. It is also to be understood that the terminology used herein is for the purpose of describing particular, embodiments only and is not intended to be limiting.

As used herein, “reaction solution” may refer to all components necessary for enzyme-based chemical transformation of β-carotene and β-cryptoxanthin. The reaction solution may typically comprise one or more buffering agent(s), salt(s), cosolvent(s), cofactor(s), detergent(s) and substrate(s) (starting material(s)).

As used herein, “reaction mixture” may refer to all components from the “reaction solution” plus the enzyme(s) and/or products (mono- or di-hydroxylated carotenoids) of the reaction.

As used herein, “buffering agents” may refer to chemicals added to water-based solutions that resist changes in pH by the action of acid-base conjugate components.

As used herein, “supernatant” may refer to the soluble liquid fraction of a sample.

As used herein, “batch reactions” may refer to a chemical or biochemical reaction performed in a closed system such as a fermenter or typical reaction flask.

As used herein, “cofactors” may refer to a non-protein chemical compound that may bind to a protein and assist with a biological chemical reaction. Non-limiting examples of cofactors may include but are not limited to NADPH and NADH.

As used herein “lysate” may refer to a fluid containing the products of cellular lysis. Lysis is the action of breaking down the cellular membrane and can be achieved by multiple mechanisms including but not limited to enzymatic, osmotic, or mechanical mechanisms.

Percent molar ratio (i.e., “% (molar ratio)”) is the molar ratio of product produced by a reaction relative to substrate used in the reaction.

In some embodiments, the product may be β-cryptoxanthin (equivalently, β-cryptoxanthin). In some embodiments, the product may be zeaxanthin.

In some embodiments, the temperature of the reaction may range from about 10° C. to about 25° C. In some embodiments, the temperature of the reaction may be about 15° C., e.g., about 10° C. to about 20° C., about 12° C. to about 18° C., or about 14° C. to about 16° C. In each case of ° C., “about” indicates ±1° C.

In some embodiments, the pH of the reaction may range from about 7.0 to about 9.0. In some embodiments, the pH of the reaction may be about 8.5 (e.g., about 7 to about 9, about 7.5 to about 9.0, about 8.0 to about 9.0, about 8.2 to about 8.8, about 8.3 to about 8.7, about 8.4 to about 8.6). In some embodiments, the pH of the reaction may be in the range of 8.2 to 8.7 or 8.3 to 8.6. In each case of pH, “about” indicates ±0.1 pH unit.

The reaction time may be varied to optimize yield or to balance yield against efficient use of resources. The reaction time may vary from 1 hour to 48 hours. In some embodiments, the time to run the reaction may range from about 5 hours to about 15 hours, e.g., about 5 hours to about 10 hours, about 5 hours to about 8 hours, about 5 hours to about 7 hours, or about 5 to about 6 hours. In each case of time, “about” indicates ±0.5 hr.

Unless otherwise specifically defined, as used herein, the term “about” refers to plus or minus 10% of the referenced number.

In some embodiments, the enzymes may be immobilized. In some embodiments, immobilized enzymes may be immobilized onto solid supports. Non-limiting examples of solid supports may include (but are not limited to) epoxy methacrylate, amino C₆ methacrylate, or microporous polymethacrylate. In further embodiments, various surface chemistries may be used for linking the immobilized enzyme to a solid surface, including but not limited to covalent, adsorption, ionic, affinity, encapsulation, or entrapment. In other embodiments, the enzymes are non-immobilized. Either immobilized or non-immobilized enzymes may be employed in batch or continuous synthesis. For example, an immobilized enzyme on a solid support may be used in a cartridge through which a reaction mixture passes, whereby an immobilized enzyme may catalyze modification of substrate to produce the product at a high titer. Alternatively, a continuous method may comprise micro mixing of enzyme solution and substrate to produce the product at a high titer, while continuously removing product, removing (e.g., recovering) substrate, or both. In some embodiments removed (e.g., recovered) substrate may be recycled to increase process efficiency and overall yield.

The starting materials and reactants for preparation of hydroxylated carotenoids from 3-carotene may be obtained from commercial sources or by readily available synthetic processes from available starting materials. For example, β-carotene is commercially available from a variety of sources e.g., Sigma-Aldrich, Tokyo Chemical Industry (TCI), etc.

Co-solvents: The reaction mixtures and reaction solutions may comprise one or more co-solvents, i.e., solvents along with water. Various co-solvents may be used in the reaction solutions and reaction mixtures to improve solubility. In some embodiments, the co-solvent may comprise a relatively hydrophobic co-solvent, or a relatively hydrophilic co-solvent, or a mixture of one or more relatively hydrophobic and one or more hydrophilic co-solvents. In some embodiments, the one or more co-solvents may comprise up to about 50% (v/v) of the reaction solution or reaction mixture, e.g., about 0.01% to about 50% (v/v), about 0.1% to about 40% (v/v), about 0.1% to about 20% (v/v), about 0.5% to about 20% (v/v), about 0.5% to about 20% (v/v), or about 0.5% to about 18% (v/v). Suitable co-solvents include, but are not limited to, e.g., tetrahydrofuran (THF), dimethylsulfoxide (DMSO), dimethylformamide (DMF), acetonitrile (ACN), or mixtures or combinations of two or more thereof. In reference to % (v/v), “about” indicates ±10% of the stated percentage (e.g., “about 0.5% (v/v)” indicates 0.5% (v/v)±0.05% (v/v) and “about 20% (v/v)” indicates 20%±2% (v/v)).

Non-ionic surfactants and detergents: The reaction mixtures and reaction solution may comprise non-ionic surfactants and detergents along with water and the other components. Various non-ionic surfactants may be used in the reaction solutions and reaction mixtures to improve solubility of substrates and products. In some embodiments, the non-ionic surfactant used may comprise 1-2% (v/v) of the reaction solution. In some embodiments the non-ionic surfactant amount may be about 4% (v/v). The non-ionic surfactants or detergents may be, e.g., Polysorbate 20, Polysorbate 80, Triton X-100, NP-40, or combinations or mixtures of two or more thereof.

Natural and synthetic redox couplers: The reaction mixtures and reaction solution may comprise reduction-oxidation coupling reagents. The reduction-oxidation coupling agents may be derived from natural or synthetic sources as cofactors. The addition of electron transfer reagents significantly increases the activity of CrtZ, allowing for an increase in product titer and extension of reaction time. In some embodiments, the redox coupler may be, e.g., methyl viologen, phenazine methosulfate, 2,6,dichloro-1,4-benzoquinone, diethyldithiocarbamate, quinone, or combinations of two or more thereof. In some embodiments, the redox coupler is present at a concentration up to 100 μM. In certain preferred embodiments of the invention, the electron transfer reagents are present at a concentration of about 1 μM to about 100 μM.

### EXAMPLES

The following is a non-limiting example of the present invention. It is to be understood that said example is not intended to limit the present invention in any way. Equivalents or substitutes are within the scope of the present invention.

Enzyme Expression and Purification: All genes were synthesized and cloned into expression plasmids and then transformed into E. coli cells for expression. Cells were grown in Terrific Broth media supplemented with 50 μg/mL kanamycin sulfate at 37° C. and 200 rpm until A₅₉₅=0.6. Cells were cooled to 18° C., protein expression was induced, and the cultures were grown for an additional 18 h. Cell pellets were collected by centrifugation, and then resuspended in a 1.8 mL lysis buffer (50 mM Tris pH 8.5, 150 mM NaCl, 10% Glycerol (v/v)) per gram of cell paste. Cell lysates were prepared by sonication and clarified by centrifugation at 11,000×g for 10 minutes, at 4° C. After clarification, lysate was kept on ice or flash frozen and stored at −80° C. for later use.

Analytical methods: For sampling, 5× volume of THE was mixed with the reaction fluid, followed by high-speed centrifugation. Samples were run on an HPLC system to examine the amount of β-cryptoxanthin and zeaxanthin present in the reaction mixture. The HPLC method was as follows: An Agilent 1200 HPLC was fitted with an Acclaim C-30 Column 250 mm×4.6 mm, 5 μm, equipped with an Acclaim C-30 guard column. The column was heated to 20° C. with the sample block being maintained at 20° C. For each sample, 10 μL was injected and the product was eluted at a flow rate of 1.0 ml/min using Methanol (solvent A) and tert-butyl-methyl ether (solvent B) with the following gradient: 80% A to 65% A for 10 min, 65% A to 10% A for 10 min, 10% A for 5 min, 10% A to 80% for 0.5 min, and 80% A for 5.5 min for column equilibration. The run time was a total of 31 minutes with zeaxanthin eluting at 7.2 mins, β-cryptoxanthin eluting at 12.2 minutes, and β-carotene eluting at 16.9 min. A diode array detector (DAD) was used for the detection of the molecule of interest at 450 nm.

Zeaxanthin Production with CrtZ Enzymes and Reaction Optimization.

As described herein, β-carotene is hydroxylated by the CrtZ enzyme (CrtZ, EC1.14.15.24) to form β-cryptoxanthin, and then β-cryptoxanthin is hydroxylated by CrtZ to form Zeaxanthin. Although heterologous CrtZ enzyme has been shown to be active in microbial hosts, a significant advancement is needed to produce industrially relevant amounts of β-cryptoxanthin and zeaxanthin. Enzymes from Table 1 were expressed and screened for activity to generate β-cryptoxanthin and zeaxanthin from β-carotene. Enzymes were initially screened for optimal values for substrate concentration, pH, temperature, detergent, co-solvent and time. The solubility of the substrate β-carotene was found to be limiting and therefore additional optimization of co-solvent type and amount in these reactions was also required. The reaction solution (1.67 mM β-carotene, 2% polysorbate 20 (v/v), 6% THE (v/v), and 0.2 mM FeSO₄) was mixed with concentrated cell lysate expressing CrtZ at 16° C. for 24 hours. P. ananatis CrtZ converted 84% (molar ratio) of 1.67 mM β-carotene substrate into zeaxanthin (˜1.4 mM, 796 mg/L).

Immobilized CrtZ to create zeaxanthin: After demonstrating and optimizing generation of Zeaxanthin from β-carotene with free enzyme, the next step is to immobilize CrtZ enzymes onto solid supports in an effort to increase stability, longevity, and catalysis. Different commercial support materials are screened for product and substrate retention, enzyme retention, and activity of the immobilized enzyme. The support collection comprises various surface chemistries for the following types of linkage: covalent, adsorption, ionic, affinity, encapsulation, and entrapment. Typically, 50 mg of resin is mixed with 4.0 mg of enzyme in buffer for 16-24 hr. at room temperature. The amount of starting material and product retained on the resin is quantified by HPLC, essentially as described hereinabove.

Following initial resin screening the best enzyme-support combination is selected for further optimization. Immobilized enzymes are again subject to various reaction conditions (changes in substrate concentration, pH, temperature, buffering agent, solvent, time) to determine the optimal activity.

CrtZ is encapsulated and the immobilized enzyme is used to convert β-carotene into zeaxanthin. Reaction solution (1.0 mM β-carotene, 2% (v/v) polysorbate 20, 6% THE (v/v), and 0.2 mM FeSO₄) is mixed with 2.5 mg immobilized enzyme at 16° C. for 24 hours. Immobilized CrtZ from P. ananatis is able to convert 1.0 mM β-carotene for a yield of 0.1 to 0.95 mM (e.g., about 0.20 to about 0.95 mM, about 0.30 to about 0.95 mM, about 0.3 to about 0.9 mM, about 0.4 to about 0.9 mM, about 0.5 to about 0.9 mM, about 0.6 to about 0.9 m, about 0.7 to about 0.9 mM, or about 0.8 to about 0.9 mM, or about 0.88 mM) Zeaxanthin (88% (molar ratio), e.g., about 0.5 g/L).

Use of Immobilized CrtZ to synthesize zeaxanthin in a continuous reactor: A reactor of 2.75″ length with a 0.125″ outside diameter and a 0.055″ internal diameter containing immobilized CrtZ enzyme (2.7 mg enzyme on 50 mg of resin) is cooled to 15° C. and equilibrated for 30 minutes with equilibration buffer by pumping it through the reactor. After this time, the substrate solution is flowed through the reactor at a flow rate of 0.4 μL/min (˜6 hr. residence time). After the solution has passed through the reactor, the fluid is collected and sampled by HPLC for the presence of β-carotene and zeaxanthin formation. The immobilized CrtZ enzyme produces zeaxanthin at a titer of about 100 to about 1000 mg/L, about 200 to about 800 mg/L, about 300 to about 700 mg/L, about 400 to about 600 mg/L, about 500 to about 600 mg/L, or about 517 mg/L in the continuous reactor.

The enzymes set forth in Table 1 below are exemplary only. One skilled in the art will appreciate that some CrtZ homologs may be categorized in the art as β-carotene hydroxylases, sterol desaturase family proteins, or fatty acid hydroxylase superfamily enzymes. The methods described herein may be practiced with any enzyme that can hydroxylate a carotene.

TABLE 1
Hydroxylase Enzyme Sequences:

| | | SEQ ID |
|---|---|---|
| Enzyme: | Sequence: | NO: |
| B-carotene hydroxylase | MLWIWNALIVFVTVIGMEVVAALAHKYIMHGW | 1 |
| Pantoea | GWGWHLSHHEPRKGAFEVNDLYAVVFAALSILL | |
| Accession: | IYLGSTGMWPLQWIGAGMTAYGLLYFMVHDGL | |
| WP_013027996.1 | VHQRWPFRYIPRKGYLKRLYMAHRMHHAVRGK | |
| | EGCVSFGFLYAPPLSKLQATLRERHGARAGAAR | |
| | DAQGGEDEPASGK | |
| ß-carotene hydroxylase | MLALWNTGIVLLTIIIMEGVATFAHKYIMHGWG | 2 |
| Enterobacteriaceae | WGWHHSHHTPRTGAFERNDLYAVVFALLAIALI | |
| Accession: | YAGSEGYWPLQWIGAGMTGYGVIYFIVHDGLVH | |
| WP_024550459 | QRWPFRYVPRRGYLRRLYMAHRLHHAVRGREG | |
| | CVSFGFIYAPPVDKLQAVLRERNGRPASAGAARG | |
| | ADRAAASSPSGKPSPASRRK | |
| Sterol desaturase | MLALYNTLIVLLTVAAMELVAALAHKYIMHGW | 3 |
| protein | GWGWHESHHEPRTSWFEVNDLYAVVFAVLAIVL | |
| Leclercia | IALGTWGIWPLQWIGAGMTLYGALYFMVHDGL | |
| Accession: | VHQRWPFRYIPRRGYLKRLYLAHRLHHAVRGKE | |
| WP_103791973.1 | DCVSFGFLYAPPVEKLQATLRQRKARRATSADA | |
| | ARARPDAASVSQNEK | |
| Sterol desaturase | MIVLYNVAIVLLTVAAMEVVAALTHKYVMHGW | 4 |
| protein | GWGWHLSHHSPRKGWFEVNDLYAVVFAGVAIL | |
| Cronobacter | LIALGAGGRWPLQWIGAGMTLYGALYFIVHDGL | |
| Accession: | VHQRWPFRYVPRRGYLKRLYLAHRLHHAVRGR | |
| WP_032983487.1 | EGCVSFGFLYAPPVAKLQAVLRERNGRPARAAA | |
| | ARAPKGEATTTRRENSQP | |
| Sterol desaturase | MNPMINALVFFATVIGMEGFAVFAHKYIMHGWG | 5 |
| protein | WGWHKSHHEPRTGWFEKNDLYAVVFAGFAIVLI | |
| Pseudomonas | ALGTQGAHPLEWIGAGMTAYGFLYFIAHDGLVH | |
| Accession: | KRWPFKYVPRNGYLKRLYQAHLMHHAVSGKER | |
| WP_122538099.1 | CVSFGFLYAPSVTRLRAQLRRLHDGPLQKSDPDV | |
| | ATGSQAARATADHESR | |
| Sterol desaturase | MEIAAALIHRYVMHGFGWGWHRSHHEPHQKRF | 6 |
| protein | ELNDLYAVVFAAIAIVLIALGTQGVWPLQWIGAG | |
| Salinicola | MTAYGLLYFIVHDGLVHKRWPFRYIPRRGYLQR | |
| Accession: | LYQAHRLHHAVKEREHGISFGFLYAPPTDKLKAE | |
| WP_071230779.1 | LRRRRPSSASEGAARDARAEDRVAVRER | |
| Sterol desaturase | MTSWTGLVVIAILVFAAMELVAWAAHKYIMHGF | 7 |
| protein | GWGWHKSHHEPHEGLFEKNDLYAVVFSILAIGLF | |
| Aureimonas | ILGSTGYPVAGAIAAGMTLYGFFYFVVHDGLVH | |
| Accession: | QRWPFRHIPHKGYVKRLVQAHRMHHAVEGREG | |
| WP_039188796.1 | CVSFGFLYAPPVDKLSEELRAAGTVKAEQAARR | |
| | AAGGARPRS | |
| Sterol desaturase | MNYLVPAALVIGTVVFMEWFAAWSHKHIMHGW | 8 |
| protein | GWRWHKSHHEPHDHALEKNDLYAVIFAVISVA | |
| Sinorhizobium | MFYIGNWYWPLWWIAVGVSVYGALYFFMHDGL | |
| Accession: | VHQRWPFRYIPRKGYLKRVYQAHRLHHAVEGR | |
| WP_028055377.1 | DGCVSFGFVYAKPADTLVKELQENKLKLSPQPP | |
| | MEEKKRDVRA | |
| Sterol desaturase | MDWFWTFMLVIAAFLGMEVFAWYAHKYIMHG | 9 |
| protein | WGWRWHKSHHEPTEGVFEKNDLYVVVFSLVVV | |
| Halomonas | GMFAVGDLYWKPLMAIASGITLYGVAYSLFHDG | |
| Accession: | MVHQRWPIRWQPKSGYLKRLVQAHRIHHAVRT | |
| WP_088701339.1 | REGAVSFGFLYAPDVRKLKKRLQQQRAAPPAGA | |
| | RHDR | |
| Sterol desaturase | MNILMPIIIVVLTVAAMEGIAYSVHRWIMHGPLG | 10 |
| protein | WGWHKSHHEETHGPFEKNDLYAVVFAVISILLFA | |
| Paracoccus | IGSAWWPWLWWVAVGASVYGVIYFIVHDGLVH | |
| Accession: | QRWPFRYVPRRGYFRRLYQAHRLHHAVEGRDD | |
| WP_103173296.1 | CVSFGFVYAPPVEDLKARLKASGVLAQRQSKHP | |
| | DAWRADRAED | |

Although there have been shown and described some preferred embodiments of the present invention, it will be readily apparent to those skilled in the art that modifications may be made thereto which do not exceed the scope of the appended claims. Therefore, the scope of the invention is only to be limited by the following claims. The figures are representative only and the claims are not limited by the dimensions of the figures. In some embodiments, descriptions of the inventions described herein employ “comprising,” “comprise,” “comprises,” or other grammatical variants (or equivalents, such as “include,” “includes,” or “including”) thereof. It is intended that “comprise” and its grammatical variants be construed as open-ended. Thus, “comprise” and its grammatical variants include embodiments that could be described as “consisting essentially of” (limited to the recited elements and only such additional elements that do not affect the unique and novel features of the invention) or “consisting of” (limited to the recited elements), and as such the written description requirement for claiming one or more embodiments of the present invention using the phrase “consisting essentially of” or “consisting of” is met by recitation of “comprise” or “include” and grammatical variants thereof. Unless otherwise specifically stated, the conjunction “or” is intended to be inclusive (e.g., “A or B” indicates “A, B, or the combination of A and B,” and “A, B or C,” indicates “A, B, C, or one or more combinations of “A and B,” “A and C,” or “A, B and C.”).

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for cell-free production of hydroxylated carotenoid, wherein the method comprises transformation of a substrate through an enzyme to hydroxylated carotenoid.

2. The method of claim 1, wherein the substrate is a carotene.

3. The method of claim 2, wherein the carotene is β-carotene.

4. The method of claim 2 or 3, wherein the hydroxylated carotenoid is a β-cryptoxanthin or zeaxanthin.

5. The methods of claims 2-4, wherein the enzyme is selected from a group consisting of: (i) β-carotene hydroxylase or a homolog thereof, (ii) sterol desaturase or a homolog thereof, (ii) fatty acid hydroxylase or a homolog thereof, and (iv) any combination thereof.

6. The methods of claims 1-5, wherein the enzyme is immobilized.

7. The methods of claims 1-5, wherein the enzyme is not immobilized.

8. The method of claim 5, wherein the β-carotene hydroxylase has an amino acid sequence at least 80% identical to the polypeptides set forth in SEQ ID NOS. 1 or 2.

9. The method of claims 2-4, wherein the enzyme is sterol desaturase and has an amino acid sequence at least 80% identical to one of the polypeptides set forth in SEQ ID NOS. 3-10.

10. The method of claim 1, wherein the hydroxylated carotenoid is produced at a concentration of at least 500 mg/L.

11. The method of claim 1, wherein the method further comprises the use of a solvent, and the solvent is selected from a group consisting of: (i) tetrahydrofuran (THF), (ii) dimethylsulfoxide (DMSO), (iii) dimethylformamide (DMF), and (iv) any combination thereof.

12. The method of claim 1, wherein the method further comprises the use of one or more non-ionic surfactants and/or detergents.

13. The method of claim 1, wherein the method comprises the use of a continuous reactor system.

14. The method of claim 1, wherein the method comprises the steps of:
in a cell-free vessel, providing a β-carotene hydroxylase enzyme (CrtZ);
adding β-carotene;
removing the hydroxylated carotenoid from the cell-free vessel.

15. The method of claim 1, wherein the hydroxylated carotenoid is a di-hydroxylated carotenoid, and the substrate is a mono-hydroxylated carotenoid.

16. The method of claim 1, wherein the method comprises the use of one or more redox coupling reagents.

17. A composition for cell-free production of hydroxylated carotenoid by enzymatic transformation of a substrate to a hydroxylated carotenoid, wherein the composition comprises a substrate and an enzyme.

18. The composition of claim 17, wherein the substrate is carotene.

19. The composition of claim 18, wherein the carotene is β-carotene.

20. The compositions of claims 17 and 18, wherein the hydroxylated carotenoid is a β-cryptoxanthin or zeaxanthin.

21. The compositions of claims 18-20, wherein the enzyme is selected from a group consisting of: (i) β-carotene hydroxylase or a homolog thereof, (ii) sterol desaturase or a homolog thereof, (ii) fatty acid hydroxylase or a homolog thereof, and (iv) any combination thereof.

22. The compositions of claims 17-21, wherein the enzyme is immobilized.

23. The compositions of claims 17-21, wherein the enzyme is not immobilized.

24. The composition of claim 21, wherein the β-carotene hydroxylase has an amino acid sequence at least 80% identical to the polypeptides set forth in SEQ ID NOS. 1 or 2.

25. The compositions of claims 18-20, wherein the enzyme is sterol desaturase and has an amino acid sequence at least 80% identical to one of the polypeptides set forth in SEQ ID NOS. 3-10.

26. The composition of claim 17, wherein the hydroxylated carotenoid is produced at a concentration of at least 500 mg/L.

27. The composition of claim 17, wherein the composition further comprises a solvent, wherein the solvent is selected from a group consisting of: (i) tetrahydrofuran (THF), (ii) dimethylsulfoxide (DMSO), (iii) dimethylformamide (DMF), and (iv) any combination thereof.

28. The composition of claim 17, wherein the composition further comprises one or more non-ionic surfactants and/or detergents.

29. The composition of claim 17, wherein the hydroxylated carotenoid is a di-hydroxylated carotenoid, and the substrate is a mono-hydroxylated carotenoid.

30. The composition of claim 17, wherein the composition further comprises one or more redox coupler reagents.


SEQUENCE LISTING (10 sequences)

SEQ ID NO: 1 - protein, 175 aa, Pantoea sp.
MLWIWNALIVFVTVIGMEVVAALAHKYIMHGWGWGWHLSHHEPRKGAFEVNDLYAVVFAALSILLIYLGSTGMWPLQWIGAGMTAYGLLYFMVHDGLVHQRWPFRYIPRKGYLKRLYMAHRMHHAVRGKEGCVSFGFLYAPPLSKLQATLRERHGARAGAARDAQGGEDEPASGK

SEQ ID NO: 2 - protein, 185 aa, Enterobacteriaceae sp.
MLALWNTGIVLLTIIIMEGVATFAHKYIMHGWGWGWHHSHHTPRTGAFERNDLYAVVFALLAIALIYAGSEGYWPLQWIGAGMTGYGVIYFIVHDGLVHQRWPFRYVPRRGYLRRLYMAHRLHHAVRGREGCVSFGFIYAPPVDKLQAVLRERNGRPASAGAARGADRAAASSPSGKPSPASRRK

SEQ ID NO: 3 - protein, 178 aa, Leclercia sp.
MLALYNTLIVLLTVAAMELVAALAHKYIMHGWGWGWHESHHEPRTSWFEVNDLYAVVFAVLAIVLIALGTWGIWPLQWIGAGMTLYGALYFMVHDGLVHQRWPFRYIPRRGYLKRLYLAHRLHHAVRGKEDCVSFGFLYAPPVEKLQATLRQRKARRATSADAARARPDAASVSQNEK

SEQ ID NO: 4 - protein, 180 aa, Cronobacter sp.
MIVLYNVAIVLLTVAAMEVVAALTHKYVMHGWGWGWHLSHHSPRKGWFEVNDLYAVVFAGVAILLIALGAGGRWPLQWIGAGMTLYGALYFIVHDGLVHQRWPFRYVPRRGYLKRLYLAHRLHHAVRGREGCVSFGFLYAPPVAKLQAVLRERNGRPARAAAARAPKGEATTTRRENSQP

SEQ ID NO: 5 - protein, 181 aa, Pseudomonas sp.
MNPMINALVFFATVIGMEGFAVFAHKYIMHGWGWGWHKSHHEPRTGWFEKNDLYAVVFAGFAIVLIALGTQGAHPLEWIGAGMTAYGFLYFIAHDGLVHKRWPFKYVPRNGYLKRLYQAHLMHHAVSGKERCVSFGFLYAPSVTRLRAQLRRLHDGPLQKSDPDVATGSQAARATADHESR

SEQ ID NO: 6 - protein, 161 aa, Salinicola sp.
MEIAAALIHRYVMHGFGWGWHRSHHEPHQKRFELNDLYAVVFAAIAIVLIALGTQGVWPLQWIGAGMTAYGLLYFIVHDGLVHKRWPFRYIPRRGYLQRLYQAHRLHHAVKEREHGISFGFLYAPPTDKLKAELRRRRPSSASEGAARDARAEDRVAVRER

SEQ ID NO: 7 - protein, 174 aa, Aureimonas sp.
MTSWTGLVVIAILVFAAMELVAWAAHKYIMHGFGWGWHKSHHEPHEGLFEKNDLYAVVFSILAIGLFILGSTGYPVAGAIAAGMTLYGFFYFVVHDGLVHQRWPFRHIPHKGYVKRLVQAHRMHHAVEGREGCVSFGFLYAPPVDKLSEELRAAGTVKAEQAARRAAGGARPRS

SEQ ID NO: 8 - protein, 171 aa, Sinorhizobium sp.
MNYLVPAALVIGTVVFMEWFAAWSHKHIMHGWGWRWHKSHHEPHDHALEKNDLYAVIFAVISVAMFYIGNWYWPLWWIAVGVSVYGALYFFMHDGLVHQRWPFRYIPRKGYLKRVYQAHRLHHAVEGRDGCVSFGFVYAKPADTLVKELQENKLKLSPQPPMEEKKRDVRA

SEQ ID NO: 9 - protein, 165 aa, Halomonas sp.
MDWFWTFMLVIAAFLGMEVFAWYAHKYIMHGWGWRWHKSHHEPTEGVFEKNDLYVVVFSLVVVGMFAVGDLYWKPLMAIASGITLYGVAYSLFHDGMVHQRWPIRWQPKSGYLKRLVQAHRIHHAVRTREGAVSFGFLYAPDVRKLKKRLQQQRAAPPAGARHDR

SEQ ID NO: 10 - protein, 175 aa, Paracoccus sp.
MNILMPIIIVVLTVAAMEGIAYSVHRWIMHGPLGWGWHKSHHEETHGPFEKNDLYAVVFAVISILLFAIGSAWWPWLWWVAVGASVYGVIYFIVHDGLVHQRWPFRYVPRRGYFRRLYQAHRLHHAVEGRDDCVSFGFVYAPPVEDLKARLKASGVLAQRQSKHPDAWRADRAED